(19) United States
(12) Patent Application Publication
(10) Pub. No.: US 2023/0368882 A1
(43) Pub. Date: Nov. 16, 2023

(54) SYSTEM AND METHOD FOR GENERATING PERSONALIZED AND COMMUNITY-BASED RECOMMENDATIONS.

(71) Applicant: JIO PLATFORMS LIMITED, Ahmedabad (IN)

(72) Inventors: Dilip KRISHNASWAMY, Bangalore (IN); Aayush BHATNAGAR, Navi Mumbai (IN); Nitish BHARDWAJ, Rishikesh (IN)

(73) Assignee: JIO PLATFORMS LIMITED, Ahmedabad (IN)

(21) Appl. No.: 18/317,620

(22) Filed: May 15, 2023

(30) Foreign Application Priority Data
May 16, 2022 (IN) 202221027968

(51) Int. Cl. G16H 20/00; G16H 40/67; G16H 10/60; A61B 5/00

(52) U.S. Cl. CPC G16H 20/00; G16H 40/67; G16H 10/60; A61B 5/6801; A61B 5/0022

(57) ABSTRACT

The present disclosure provides a system and a method for providing personalized and community-based recommendations. The system is configured with one or more primary sensors and a wearable device to monitor multiple health parameters from a user. The wearable device is configured with one or more secondary sensors to monitor additional health parameters from the user. Further, the system uses artificial intelligence (AI) to generate a personalized model or a digital twin based on the inputs from the one or more primary sensors and the wearable device. The generated personalized model continuously monitors the inputs and triggers an emergency service upon a significant variation in the inputs. Further, the system provides various micro services to facilitate personalized and community based recommendations. The system utilizes a blockchain network to generate smart contracts and reward the user based on the inputs from the digital twin model.

### FIELD OF INVENTION

The embodiments of the present disclosure generally relate to systems and methods for community health predictions using artificial intelligence (AI) and blockchain technology. More particularly, the present disclosure relates to a system and a method for generating personalized and community-based recommendations.

### BACKGROUND OF INVENTION

The following description of the related art is intended to provide background information pertaining to the field of the disclosure. This section may include certain aspects of the art that may be related to various features of the present disclosure. However, it should be appreciated that this section is used only to enhance the understanding of the reader with respect to the present disclosure, and not as admissions of the prior art.

Community health assessment includes health informatics, user data, supplies, and other procedures. As the world is facing multiple healthcare challenges, the existing digital healthcare technology needs to be upgraded. Thus, healthcare deliver solutions need to be personalized and monitored to cater to various individuals, community needs, and mitigate clinical risk during diagnosis. Further, new guidelines, protocols, and clinical pathways may be provided with an advanced technology to promote higher collaboration among healthcare systems.

There is, therefore, a need in the art to provide a system and a method that can mitigate the problems associated with the prior arts.

### OBJECTS OF THE INVENTION

Some of the objects of the present disclosure, which at least one embodiment herein satisfies are listed herein below.

It is an object of the present disclosure to provide a system and a method that uses artificial intelligence (AI) to generate a trained model for generating community based recommendations based on an input provided by a user.

It is an object of the present disclosure to provide a personal digital twin based on AI and block chain technology, where the personal digital twin model is used to generate personalized recommendations.

It is an object of the present disclosure to provide a system and a method where the personalized digital twin is powered by AI and blockchain technology to enable efficient decision making and provide accurate predictions/alerts based on personalized and community based models.

It is an object of the present disclosure to provide a system and a method that generates an ecosystem powered by the blockchain technology that rewards users for sharing the inputs and provides efficient community based services.

It is an object of the present disclosure to provide a system and a method that provides a health monitoring device equipped with various sensors to measure multiple data parameters from the user.

It is an object of the present disclosure to provide a system and a method that enables measurement of various health parameters from the user via a wearable device.

It is an object of the present disclosure to provide a system and a method that is secure, transparent, user-friendly, and ensures that users are always in control of their data.

### SUMMARY

This section is provided to introduce certain objects and aspects of the present disclosure in a simplified form that are further described below in the detailed description. This summary is not intended to identify the key features or the scope of the claimed subject matter.

In an aspect, the present disclosure relates to a system for generating personalized and community-based recommendations. The system may include a processor and a memory operatively coupled to the processor that stores instructions to be executed by the processor. The processor may receive one or more data parameters via one or more primary sensors. The one or more primary sensors may be communicatively coupled to the processor. The received one or more data parameters may be based on one or more inputs provided by a user via a computing device. The processor may receive one or more health parameters from the user via a wearable device. The wearable device may be adaptively secured to the user and connected to the processor via a network. The processor may generate via an artificial intelligence (AI) engine, a personalized model based on the received one or more data parameters and the received one or more health parameters. The processor may generate the personalized and community-based recommendations based on the generated personalized model.

In an embodiment, the one or more primary sensors may include at least one of a temperature sensor, a blood pressure sensor, an oxygen saturation sensor, and a heart-rate sensor.

In an embodiment, the wearable device may be configured with one or more secondary sensors that may include at least one of a temperature sensor, a blood pressure sensor, an oxygen saturation sensor, a heart rate sensor, a motion sensor, a camera, a global positioning system (GPS), and a microphone.

In an embodiment, the processor may be configured with one or more micro services to provide inputs to the user based on the generated personalized and community-based recommendations.

In an embodiment, the one or more micro services may include at least one of a blockchain ledger and an internet of things (IoT) service.

In an embodiment, the one or more micro services may be configured to update the generated personalized model and aid in the generation of the personalized and community-based recommendations.

In an embodiment, the processor may be configured with a vault service to secure the generated personalized model to enable the user to access the vault service via the computing device.

In an embodiment, the processor may be configured with an identification service (IS) that enables a mapping between the user and the generated personalized model.

In an embodiment, the processor may be communicatively coupled to a blockchain network that may generate a reward based on the one or more data parameters and the generated personalized model.

In an embodiment, the blockchain network may include at least one of a distributed consensus, a smart contract, a wallet service, and a distributed ledger.

In an aspect, the present disclosure relates to a method for generating personalized and community-based recommendations. The method may include receiving, by a processor associated with a system, one or more data parameters via one or more primary sensors. The one or more primary sensors may be communicatively coupled to the processor. The received one or more data parameters may be based on one or more inputs provided by a user via a computing device. The method may include receiving, by the processor, one or more health parameters from the user via a wearable device. The wearable device may be adaptively secured to the user. The method may include generating, by the processor, via an AI engine, a personalized model based on the received one or more data parameters and the received one or more health parameters. The method may include generating, by the processor, the personalized and community-based recommendations based on the generated personalized model.

In an embodiment, the method may include providing, by the processor, via one or more micro-services, inputs to the user based on the generated personalized and community-based recommendations.

In an embodiment, the one or more micro services may include at least one of a blockchain ledger and an IoT service.

In an embodiment, the method may include updating, by the processor via one or more micro services, the generated personalized model and aiding in the generation of the personalized and community-based recommendations.

In an embodiment, the method may include securing, by the processor, the generated personalized model via a configured vault service to enable the user to access the vault service via the computing device.

In an embodiment, the method may include mapping, by the processor, the user with the generated personalized model via a configured IS.

In an embodiment, the method may include generating, by the processor, via a blockchain network, a reward based on the one or more data parameters and the generated personalized model.

In an aspect, a user equipment (UE) for receiving personalized and community-based recommendations may include one or more processors communicatively coupled to a processor associated with a system. The one or more processors may be coupled with a memory. The memory may store instructions to be executed by the one or more processors that may cause the one or more processors to transmit one or more data parameters via one or more primary sensors to the processor via a network, and receive the personalized and community-based recommendations from the system. The processor may be configured to receive the one or more data parameters from the UE. The received one or more data parameters may be based on one or more inputs provided by a user via the UE. The processor may receive one or more health parameters from the user via a wearable device. The wearable device may be adaptively secured to the user and connected to the processor via the network. The processor may generate, via an AI engine, a personalized model based on the received one or more data parameters and the received one or more health parameters. The processor may generate the personalized and community-based recommendations based on the generated personalized model.

### BRIEF DESCRIPTION OF DRAWINGS

The accompanying drawings, which are incorporated herein, and constitute a part of this disclosure, illustrate exemplary embodiments of the disclosed methods and systems which like reference numerals refer to the same parts throughout the different drawings. Components in the drawings are not necessarily to scale, emphasis instead being placed upon clearly illustrating the principles of the present disclosure. Some drawings may indicate the components using block diagrams and may not represent the internal circuitry of each component. It will be appreciated by those skilled in the art that disclosure of such drawings includes the disclosure of electrical components, electronic components, or circuitry commonly used to implement such components.

FIG. 1 illustrates an exemplary network architecture (100) of a proposed system (110), in accordance with an embodiment of the present disclosure.

FIG. 2 illustrates an exemplary block diagram (200) of a proposed system (110), in accordance with an embodiment of the present disclosure.

FIG. 3 illustrates an exemplary system architecture (300), in accordance with an embodiment of the present disclosure.

FIG. 4 illustrates an exemplary flow diagram (400) for implementing the system (110) to generate personalized and community-based recommendations, in accordance with an embodiment of the present disclosure.

FIG. 5 illustrates an exemplary computer system (500) in which or with which embodiments of the present disclosure may be implemented.

The foregoing shall be more apparent from the following more detailed description of the disclosure.

### BRIEF DESCRIPTION OF THE INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the disclosure as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

Also, it is noted that individual embodiments may be described as a process that is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive in a manner similar to the term “comprising” as an open transition word without precluding any additional or other elements.

Reference throughout this specification to “one embodiment” or “an embodiment” or “an instance” or “one instance” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the disclosure. As used herein, the singular forms “a”, “an”, and “the” are intended to include the plural forms as well, unless the context indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

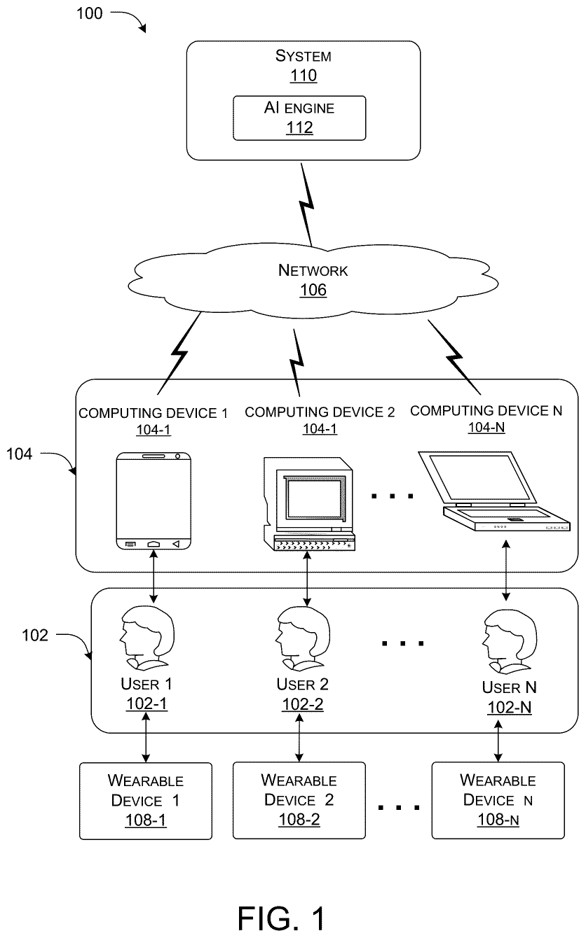

The various embodiments throughout the disclosure will be explained in more detail with reference to FIGS. 1-5.

FIG. 1 illustrates an exemplary network architecture (100) of a proposed system (110), in accordance with an embodiment of the present disclosure.

As illustrated in FIG. 1, the network architecture (100) may include a system (110). The system (110) may be connected to one or more computing devices (104-1, 104-2 . . . 104-N) via a network (106). The one or more computing devices (104-1, 104-2 . . . 104-N) may be interchangeably specified as a user equipment (UE) (104) and be operated by one or more users (102-1, 102-2 . . . 102-N). Further, the one or more users (102-1, 102-2 . . . 102-N) may be interchangeably referred as a user (102) or users (102). In an embodiment, the system (110) may receive one or more data parameters via one or more primary sensors configured with the system (110). The received one or more data parameters may be based on one or more inputs provided by the user (102) via the computing devices (104). In an embodiment, the users (102) may be adaptively secured with a wearable device (108-1, 108-2 . . . 108-N) to measure one or more health parameters associated with the users (102). The wearable device (108) may be connected to the system (110) via the network (106).

In an embodiment, the one or more inputs may include, but not limited to, health data, financial data, travel itineraries, etc. Further, the one or more inputs may be provided by various communities that include, but not be limited to, hospitals, banks, travel agencies, and retail markets.

In an embodiment, the one or more data parameters may include, but not limited to, qualitative data and quantitative data. Further, the one or more data parameters may include data parameters from operational systems, multimedia, business partnerships, financial transactions, social media, health assistance systems, mobile application, and Internet of Things (IoT) applications.

Further, in an embodiment, the one or more health parameters may include, but not limited to, heart rate, pulse rate, breathing rate, blood flow, heartbeat signatures, cardio-pulmonary health, organ health, metabolism, electrolyte type and/or concentration, physical activity, caloric intake, caloric metabolism, blood metabolite levels or ratios, stress level indicators, drug dosage and/or dosimetry, physiological drug reactions, position and/or balance, body strain, neurological functioning, brain activity, blood pressure, cranial pressure, and hydration levels.

In an embodiment, system (110) may include an artificial intelligence (AI) engine (112) to generate a personalized model based on the one or more health parameters and the one or more data parameters. The personalized model may generate and provide personalized and community-based recommendations to the users (102). In an embodiment, the generated personalized model may include one or more digital twins that may trigger emergency services based on a variation in the one or more data parameters and the one or more health parameters associated with the users (102).

In an embodiment, the computing devices (104) may include, but not be limited to, a mobile, a laptop, etc. Further, the computing devices (104) may include a smartphone, virtual reality (VR) devices, augmented reality (AR) devices, a general-purpose computer, desktop, personal digital assistant, tablet computer, and a mainframe computer. Additionally, input devices for receiving input from the user (102) such as a touch pad, touch-enabled screen, electronic pen, and the like may be used. A person of ordinary skill in the art will appreciate that the computing devices (104) may not be restricted to the mentioned devices and various other devices may be used.

In an embodiment, the network (106) may include, by way of example but not limitation, at least a portion of one or more networks having one or more nodes that transmit, receive, forward, generate, buffer, store, route, switch, process, or a combination thereof, etc. one or more messages, packets, signals, waves, voltage or current levels, some combination thereof, or so forth. The network (106) may also include, by way of example but not limitation, one or more of a wireless network, a wired network, an internet, an intranet, a public network, a private network, a packet-switched network, a circuit-switched network, an ad hoc network, an infrastructure network, a Public-Switched Telephone Network (PSTN), a cable network, a cellular network, a satellite network, a fiber optic network, or some combination thereof.

In an embodiment, the system (110) may include an edge device that may assist with healthcare monitoring services. The edge device may include one or more primary sensors for measurement of, but not limited to, temperature, blood pressure, oxygen saturation, and heart-rate of the user (102). The edge device may be connected to the computing device (104) via Bluetooth and may further include an application that may be accessed by the users (102) for providing the one or more data parameters. The one or more data parameters may include additional parameters provided by the users (102) from operational systems, multimedia, business partnerships, financial transactions, social media, health assistance systems, mobile application, and IoT applications. In an embodiment, the user (102) may choose when to connect to the edge device and upload the measurements, i.e. data parameters from the one or more primary sensors to the application.

In an embodiment, the wearable device (108) may be configured with one or more secondary sensors to measure health parameters such as, but not limited to, body temperature, blood pressure, oxygen saturation level, heart rate, motion sensors such as an accelerometer and a gyroscope, a camera (used only in an emergency), global positioning system (GPS) sensor (turned on for emergency uses), and a microphone. The wearable device (108) may monitor health of the users (102) and enable personalized or participatory community healthcare services. Further, the wearable device (108) may provide support for payment services for travel or retail, rewards for travel/retail use-cases, etc. The wearable device (108) may include a cellular modem to connect directly to a fifth generation (5G) network as well as a Bluetooth low energy (BLE) modem and enable the computing device (104) for 5G/fourth generation (4G) connectivity. Based on a proximity to the computing device (104) and a BLE mode, the wearable device (108) may shut off its direct cellular connection to save energy (Default Paired 5G Connection Mode).

In an embodiment, the system (110) may include one or more micro services to provide inputs to the user (102) based on the generated personalized model. The one or more micro services may include, but not limited to, a blockchain ledger and an IoT service. The micro services architecture may support personalized community services and calculate rewards. Various micro services may be invoked as needed for different verticals in different contexts. These micro services may relate to augmenting the personalized digital twin model or a community digital twin model and record events on the blockchain ledger. Further, the micro services may provide relevant IoT services in different contexts and may be scaled upon demand.

In an embodiment, the system (110) may generate personalized and community-based recommendations, which recommend generic services based on a community data. An aggregated data may be used without any personal information to train one or more community based models. The users (102) may select the type of data they are willing to share and the one or more community based models may be trained on this data. In an embodiment, the users (102) may be rewarded for sharing their data and rewards may be calculated by a smart contract execution.

In an embodiment, the system (110) may include a vault service to secure the generated personalized model to enable the user (102) to access the vault service via the computing device (104). The vault service may provide a secure storage to replicate any personalized model which is trained on user data using a highly efficient machine learning algorithm. This personalized model may act as a digital twin and may provide accurate suggestions to the user (102). The user (102) may opt to have a personal digital health vault for their personal digital twin model/generated personalized model and the personal digital twin model may be re-used when switching computing devices (104). Further, the vault service may prevent any overlap with other trained models and store the user's (102) personal digital twin model securely.

In an embodiment, the system (110) may include an identification service (IS) that enables a mapping between the user (102) and the generated personalized model. The IS may be used for managing the user's (102) digital identity. The digital identity may be essential to map the user (102) and their corresponding vault storage for storing the generated personalized model.

In an embodiment, the system (110) may be communicatively coupled to a blockchain network that may generate a reward based on the data parameters and the generated personalized model. Rewards may be provided for different events occurring across verticals. Rewards may include a reward for utilizing public transport or green electric vehicles. Rewards may include a reward for retail purchases and sharing anonymized information related to such purchases. Rewards may include a reward for sharing anonymized healthcare information. Also, rewards may include a reward for good driving behaviour.

In an embodiment, the generated personalized model may include a predefined criteria to reward the user (102) based on the ecosystem business requirements. As soon as any collected dataset matches the ecosystem business requirements criteria, the system (110) may invoke a smart contract for rewarding the user (102). The smart contract may calculate the rewards and ensure that a consensus is achieved with other nodes of the network (106) before the information is written to the blockchain ledger.

Although FIG. 1 shows exemplary components of the network architecture (100), in other embodiments, the network architecture (100) may include fewer components, different components, differently arranged components, or additional functional components than depicted in FIG. 1. Additionally, or alternatively, one or more components of the network architecture (100) may perform functions described as being performed by one or more other components of the network architecture (100).

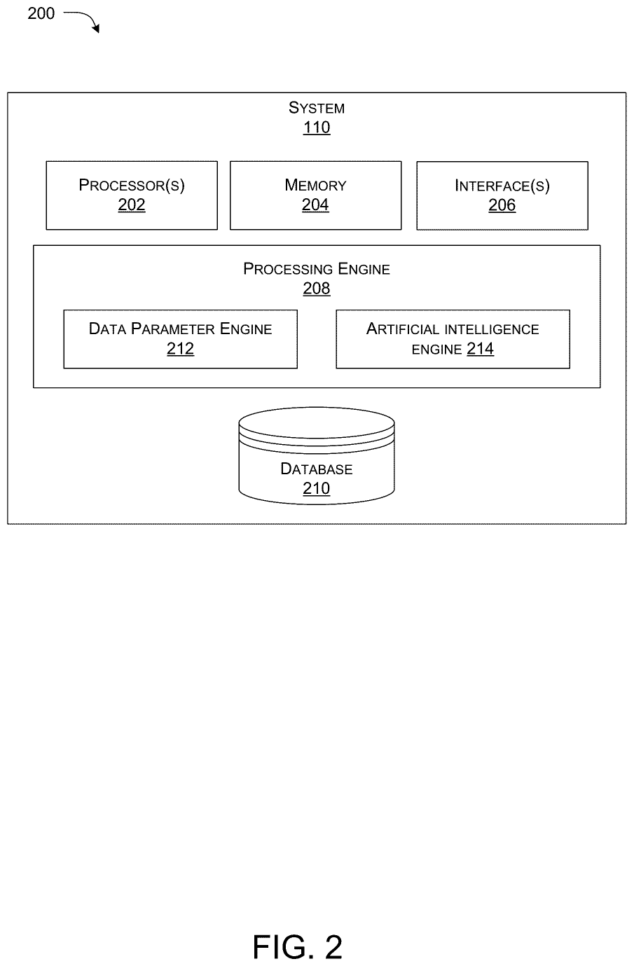

FIG. 2 illustrates an exemplary block diagram (200) of a proposed system (110), in accordance with an embodiment of the present disclosure.

Referring to FIG. 2, the system (110) may comprise one or more processor(s) (202) that may be implemented as one or more microprocessors, microcomputers, microcontrollers, digital signal processors, central processing units, logic circuitries, and/or any devices that process data based on operational instructions. Among other capabilities, the one or more processor(s) (202) may be configured to fetch and execute computer-readable instructions stored in a memory (204) of the system (110). The memory (204) may be configured to store one or more computer-readable instructions or routines in a non-transitory computer readable storage medium, which may be fetched and executed to create or share data packets over a network service. The memory (204) may comprise any non-transitory storage device including, for example, volatile memory such as random-access memory (RAM), or non-volatile memory such as erasable programmable read only memory (EPROM), flash memory, and the like.

In an embodiment, the system (110) may include an interface(s) (206). The interface(s) (206) may comprise a variety of interfaces, for example, interfaces for data input and output (I/O) devices, storage devices, and the like. The interface(s) (206) may also provide a communication pathway for one or more components of the system (110). Examples of such components include, but are not limited to, processing engine(s) (208) and a database (210), where the processing engine(s) (208) may include, but not be limited to, a data parameter engine (212) and an AI engine (214). A person with ordinary skill in the art may understand that the AI engine (214) may be similar to the AI engine (112) of FIG. 1 in its functionality.

In an embodiment, the processing engine(s) (208) may be implemented as a combination of hardware and programming (for example, programmable instructions) to implement one or more functionalities of the processing engine(s) (208). In examples described herein, such combinations of hardware and programming may be implemented in several different ways. For example, the programming for the processing engine(s) (208) may be processor-executable instructions stored on a non-transitory machine-readable storage medium and the hardware for the processing engine(s) (208) may comprise a processing resource (for example, one or more processors), to execute such instructions. In the present examples, the machine-readable storage medium may store instructions that, when executed by the processing resource, implement the processing engine(s) (208). In such examples, the system (110) may comprise the machine-readable storage medium storing the instructions and the processing resource to execute the instructions, or the machine-readable storage medium may be separate but accessible to the system (110) and the processing resource. In other examples, the processing engine(s) (208) may be implemented by electronic circuitry.

In an embodiment, the processor (202) may receive one or more data parameters from one or more primary sensors via the data parameter engine (212). The processor (202) may store the one or more data parameters in the database (210). The one or more primary sensors may be communicatively coupled to the processor (202). The one or more primary sensors may include, but not limited to, a temperature sensor, a blood pressure sensor, an oxygen saturation sensor, and a heart-rate sensor. The received one or more data parameters may be based on one or more inputs provided by the user (102) via the computing device (104). The processor (202) may receive one or more health parameters from the user (102) via the wearable device (108). The wearable device (108) may be adaptively secured to the user (102) and connected to the processor (202) via the network (106). The wearable device (108) may be configured with one or more secondary sensors that may include, but not limited to, a temperature sensor, a blood pressure sensor, an oxygen saturation sensor, a heart rate sensor, a motion sensor, a camera, a GPS, and a microphone.

In an embodiment, the processor (202) may generate, via the AI engine (214), a personalized model based on the received one or more data parameters and the received one or more health parameters. The processor (202) may generate personalized and community-based recommendations based on the generated personalized model.

In an embodiment, the processor (202) may be configured with one or more micro services to provide inputs to the user (102) based on the generated personalized and community-based recommendations. The one or more micro services may include, but not limited to, a blockchain ledger and an IoT service. The one or more micro services may be configured to update the generated personalized model and aid in the generation of the personalized and community-based recommendations.

In an embodiment, the processor (202) may be configured with a vault service to secure the generated personalized model to enable the user (102) to access the vault service via the computing device (104).

In an embodiment, the processor (202) may be configured with an IS that enables a mapping between the user (102) and the generated personalized model.

In an embodiment, the processor (202) may be communicatively coupled to a blockchain network that may generate a reward based on the data parameters and the generated personalized model. The blockchain network may include, but not limited to, a distributed consensus, a smart contract, a wallet service, and a distributed ledger.

Although FIG. 2 shows exemplary components of the system (110), in other embodiments, the system (110) may include fewer components, different components, differently arranged components, or additional functional components than depicted in FIG. 2. Additionally, or alternatively, one or more components of the system (110) may perform functions described as being performed by one or more other components of the system (110).

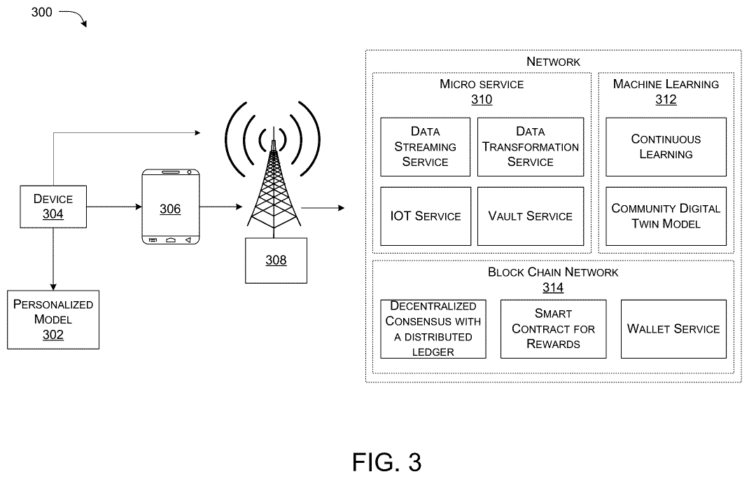

FIG. 3 illustrates an exemplary system architecture (300), in accordance with an embodiment of the present disclosure.

As illustrated in FIG. 3, an edge device (304) may be connect to a computing device (306) via BLE or may directly connect to a network (308). A person of ordinary skill in the art will understand that the computing device (306) and the network (308) may be similar to the respective computing device (104) and the network (106) of FIG. 1 in their functionality, and hence may not be described in detail again for the sake of brevity.

In an embodiment, the edge device (304) may generate a personalized model (302) based on the inputs provided by a user (102). In an embodiment, the network (308) may include different micro services (310) to facilitate personalized and community-based services. These micro services (310) may streamline shared data from the users (102) and transform in real-time to train and improve the personalized model (302). The micro services (310) may be scaled up/down based on the load on the network (308) and may be deployed to any cloud environment. Further, the micro services (310) may include, but not limited to, a data streaming service, a data transformation service, an IoT service, and a vault service.

In an embodiment, the personalized model (302) may be trained using machine learning (312), where a reinforcement learning technique may be employed. The personalized model (302) may be trained based on the shared data and may improve with every shared dataset. The personalized model (302) may provide generalized suggestions to the user (102) based on, but not limited to, geolocation, age, and other generalized factors. Further, the personalized model (302) may read the transformed data set using the data transformation service and process the transformed data to improve itself. The machine learning (312) may include continuous learning and community digital twin models.

In an embodiment, a blockchain network (314) may include, but not limited to, a decentralized consensus, a smart contract, a wallet service, and a distributed ledger. Smart contracts may be used to calculate the rewards based on inputs from the personalized model (302). Further, the distributed ledger may be updated based on the rewards calculated for the users (102).

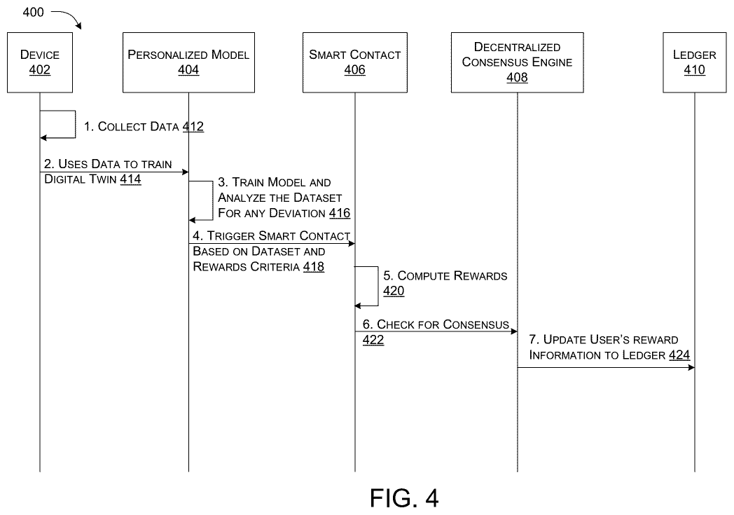

FIG. 4 illustrates an exemplary flow diagram (400) for implementing the system (110) to generate personalized and community-based recommendations, in accordance with an embodiment of the present disclosure. A person skilled in the art may understand that the device (402) of FIG. 4 may be similar to the wearable device (108) in its functionality. Further, the ledger (410) of FIG. 4 may be similar to the distributed ledger, part of the blockchain network (314) of FIG. 3 in its functionality.

As illustrated in FIG. 4, the following steps may be implemented by the system (110) for generating the personalized and community-based recommendations.

At step 412: The device (402) may collect data from users (102).

At step 414: The device (402) may utilize the data to train a digital twin.

At step 416: A personalized model (404) may analyze the digital twin and the data for any deviation.

At step 418: The personalized model (404) may trigger a smart contract (406) based on the dataset and a rewards criteria.

At step 420: The smart contract (406) may compute the rewards.

At step 422: The smart contract (406) may check for consensus while sending the information to a decentralized consensus engine (408).

At step 424: The decentralized consensus engine (408) may update the user's (102) reward information on a ledger (410).

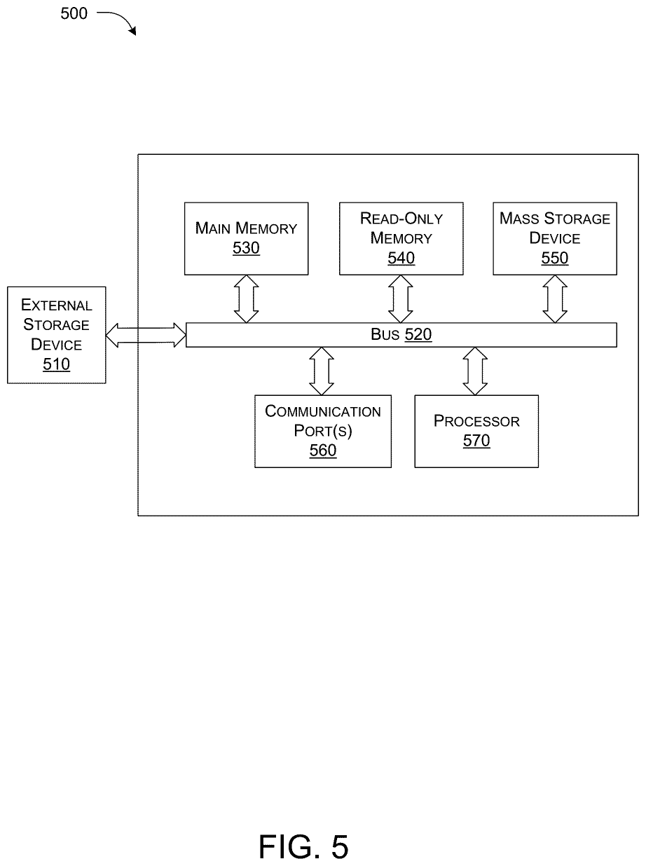

FIG. 5 illustrates an exemplary computer system (500) in which or with which embodiments of the present disclosure may be implemented. In an embodiment, the system (110) may be implemented as the computer system (500).

As shown in FIG. 5, the computer system (500) may include an external storage device (510), a bus (520), a main memory (530), a read-only memory (540), a mass storage device (550), a communication port(s) (560), and a processor (570). A person skilled in the art will appreciate that the computer system (500) may include more than one processor and communication ports. The processor (570) may include various modules associated with embodiments of the present disclosure. The communication port(s) (560) may be any of an RS-232 port for use with a modem-based dialup connection, a 10/100 Ethernet port, a Gigabit or 10 Gigabit port using copper or fiber, a serial port, a parallel port, or other existing or future ports. The communication ports(s) (560) may be chosen depending on a network, such as a Local Area Network (LAN), Wide Area Network (WAN), or any network to which the computer system (500) connects.

In an embodiment, the main memory (530) may be Random Access Memory (RAM), or any other dynamic storage device commonly known in the art. The read-only memory (540) may be any static storage device(s) e.g., but not limited to, a Programmable Read Only Memory (PROM) chip for storing static information e.g., start-up or basic input/output system (BIOS) instructions for the processor (570). The mass storage device (550) may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces).

In an embodiment, the bus (520) may communicatively couple the processor(s) (570) with the other memory, storage, and communication blocks. The bus (520) may be, e.g. a Peripheral Component Interconnect PCI)/PCI Extended (PCI-X) bus, Small Computer System Interface (SCSI), universal serial bus (USB), or the like, for connecting expansion cards, drives, and other subsystems as well as other buses, such a front side bus (FSB), which connects the processor (570) to the computer system (500).

In another embodiment, operator and administrative interfaces, e.g., a display, keyboard, and cursor control device may also be coupled to the bus (520) to support direct operator interaction with the computer system (500). Other operator and administrative interfaces can be provided through network connections connected through the communication port(s) (560). Components described above are meant only to exemplify various possibilities. In no way should the aforementioned exemplary computer system (500) limit the scope of the present disclosure.

While considerable emphasis has been placed herein on the preferred embodiments, it will be appreciated that many embodiments can be made and that many changes can be made in the preferred embodiments without departing from the principles of the disclosure. These and other changes in the preferred embodiments of the disclosure will be apparent to those skilled in the art from the disclosure herein, whereby it is to be distinctly understood that the foregoing descriptive matter is to be implemented merely as illustrative of the disclosure and not as a limitation.

### Advantages of the Invention

The present disclosure provides a system and a method that uses artificial intelligence (AI) to generate a trained model for generating community-based recommendations based on an input provided by a user.

The present disclosure provides a personal digital twin based on AI and block chain technology, where the personal digital twin model is used to generate personalized recommendations.

The present disclosure provides a system and a method where the personalized digital twin is powered by AI and blockchain technology to enable efficient decision making and provide accurate predictions/alerts based on personalized and community-based models.

The present disclosure provides a system and a method that generates an ecosystem powered by blockchain technology that rewards users for sharing the inputs and provides efficient community-based services.

The present disclosure provides a system and a method that provides a health monitoring device equipped with various sensors to measure multiple data parameters from the user.

The present disclosure provides a system and a method that enables measurement of various health parameters from the user via a wearable device.

The present disclosure provides a system and a method that is secure, transparent, user-friendly, and ensures that users are always in control of their data.

We claim:

1. A system (110) for generating personalized and community-based recommendations, the system (110) comprising:
a processor (202); and
a memory (204) operatively coupled with the processor (202), wherein said memory (204) stores instructions, which when executed by the processor (202), causes the processor (202) to:
receive one or more data parameters via one or more primary sensors communicatively coupled to the processor (202), wherein the received one or more data parameters are based on one or more inputs provided by a user (102) via a computing device (104);
receive one or more health parameters from the user (102) via a wearable device (108), wherein the wearable device (108) is adaptively secured to the user (102) and connected to the processor (202) via a network (106);
generate, via an artificial intelligence (AI) engine (112), a personalized model based on the received one or more data parameters and the received one or more health parameters; and
generate the personalized and community-based recommendations based on the generated personalized model.

2. The system (110) as claimed in claim 1, wherein the one or more primary sensors comprise at least one of: a temperature sensor, a blood pressure sensor, an oxygen saturation sensor, and a heart-rate sensor.

3. The system (110) as claimed in claim 1, wherein the wearable device (108) is configured with one or more secondary sensors that comprise at least one of: a temperature sensor, a blood pressure sensor, an oxygen saturation sensor, a heart rate sensor, a motion sensor, a camera, a global positioning system (GPS), and a microphone.

4. The system (110) as claimed in claim 1, wherein the processor (202) is configured with one or more micro services to provide inputs to the user (102) based on the generated personalized and community-based recommendations.

5. The system (110) as claimed in claim 4, wherein the one or more micro services comprise at least one of: a blockchain ledger and an internet of things (IoT) service.

6. The system (110) as claimed in claim 4, wherein the one or more micro services are configured to update the generated personalized model and aid in the generation of the personalized and community-based recommendations.

7. The system (110) as claimed in claim 1, wherein the processor (202) is configured with a vault service to secure the generated personalized model to enable the user (102) to access the vault service via the computing device (104).

8. The system (110) as claimed in claim 1, wherein the processor (202) is configured with an identification service (IS) that enables a mapping between the user (102) and the generated personalized model.

9. The system (110) as claimed in claim 1, wherein the processor (202) is communicatively coupled to a blockchain network that generates a reward based on the one or more data parameters and the generated personalized model.

10. The system (110) as claimed in claim 9, wherein the blockchain network comprises at least one of: a distributed consensus, a smart contract, a wallet service, and a distributed ledger.

11. A method for generating personalized and community-based recommendations, the method comprising:
receiving, by a processor (202), one or more data parameters via one or more primary sensors communicatively coupled to the processor (202), wherein the received one or more data parameters are based on one or more inputs provided by a user (102) via a computing device (104);
receiving, by the processor (202), one or more health parameters from the user (102) via a wearable device (108), wherein the wearable device (108) is adaptively secured to the user (102);
generating, by the processor (202) via an artificial intelligence (AI) engine (112), a personalized model based on the received one or more data parameters and the received one or more health parameters; and
generating, by the processor (202), the personalized and community-based recommendations based on the generated personalized model.

12. The method as claimed in claim 11, comprising providing, by the processor (202) via one or more micro services, inputs to the user (102) based on the generated personalized and community-based recommendations.

13. The method as claimed in claim 12, wherein the one or more micro services comprise at least one of: a blockchain ledger and an internet of things (IoT) service.

14. The method as claimed in claim 12, comprising updating, by the processor (202) via the one or more micro services, the generated personalized model and aiding in the generation of the personalized and community-based recommendations.

15. The method as claimed in claim 11, comprising securing, by the processor (202), the generated personalized model via a configured vault service to enable the user (102) to access the vault service via the computing device (104).

16. The method as claimed in claim 11, comprising mapping, by the processor (202), the user (102) with the generated personalized model via a configured identification service (IS).

17. The method as claimed in claim 11, comprising generating, by the processor (202) via a blockchain network, a reward based on the one or more data parameters and the generated personalized model.

18. A user equipment (UE) (104) for receiving personalized and community-based recommendations, the UE (104) comprising:
one or more processors communicatively coupled to a processor (202) associated with a system (110), wherein the one or more processors are coupled with a memory, and wherein said memory stores instructions, which when executed by the one or more processors, cause the one or more processors to:
transmit one or more data parameters via one or more primary sensors to the processor (202) via a network (106); and
receive the personalized and community-based recommendations from the system (110),
wherein the processor (202) is configured to:
receive the one or more data parameters from the UE (104),
wherein the received one or more data parameters are based on one or more inputs provided by a user (102) via the UE (104);
receive one or more health parameters from the user (102) via a wearable device (108), wherein the wearable device (108) is adaptively secured to the user (102) and connected to the processor (202) via the network (106);
generate, via an artificial intelligence (AI) engine (112), a personalized model based on the received one or more data parameters and the received one or more health parameters; and
generate the personalized and community-based recommendations based on the generated personalized model.
